SEQUENCE LISTING (44 sequences)

SEQ ID NO: 1 - protein, 25 aa, Homo sapiens
PGPQNFPPQNMFEFPPHLSPPLLPP

SEQ ID NO: 2 - protein, 25 aa, Homo sapiens
GAQEEPQVEPLDFSLPKQQGELLER

SEQ ID NO: 3 - protein, 25 aa, Homo sapiens
AVFAGSDDPFATPLSMSEMDRRNDA

SEQ ID NO: 4 - protein, 25 aa, Homo sapiens
HSGQNHLKEMAISVLEARACAAAGQ

SEQ ID NO: 5 - protein, 25 aa, Homo sapiens
ILPQAPSGPSYATYLQPAQAQMLTP

SEQ ID NO: 6 - protein, 19 aa, Homo sapiens
MSYAEKSDEITKDEWMEKL

SEQ ID NO: 7 - protein, 25 aa, Homo sapiens
GAGKGKYYAVNFSMRDGIDDESYGQ

SEQ ID NO: 8 - protein, 25 aa, Homo sapiens
YRGADKLCRKASSVKLVKTSPELSE

SEQ ID NO: 9 - protein, 25 aa, Homo sapiens
DSNLQARLTSYETLKKSLSKIREES

SEQ ID NO: 10 - protein, 25 aa, Homo sapiens
HSFIHAAMGMAVTWCAAIMTKGQYS

SEQ ID NO: 11 - protein, 25 aa, Homo sapiens
LRTAAYVNAIEKIFKVYNEAGVTFT

SEQ ID NO: 12 - protein, 25 aa, Homo sapiens
FEGSLAKNLSLNFQAVKENLYYEVG

SEQ ID NO: 13 - protein, 25 aa, Homo sapiens
DPRAAYFRQAENDMYIRMALLATVL

SEQ ID NO: 14 - protein, 25 aa, Homo sapiens
LRSQMVMKMREYFCNLHGFVDIETP

SEQ ID NO: 15 - protein, 25 aa, Homo sapiens
DLLAFERKLDQTVMRKRLDIQEALK

SEQ ID NO: 16 - protein, 25 aa, Homo sapiens
IKREKCWKDATYPESFHTLESVPAT

SEQ ID NO: 17 - protein, 25 aa, Homo sapiens
GRSSQVYFTINVNLDLSEAAVVTFS

SEQ ID NO: 18 - protein, 25 aa, Homo sapiens
KPLRRNNSYTSYIMAICGMPLDSFR

SEQ ID NO: 19 - protein, 25 aa, Homo sapiens
TTCLAVGGLDVKFQEAALRAAPDIL

SEQ ID NO: 20 - protein, 25 aa, Homo sapiens
IYEFDYHLYGQNITMIMTSVSGHLL

SEQ ID NO: 21 - protein, 25 aa, Homo sapiens
PDSFSIPYLTALDDLLGTALLALSF

SEQ ID NO: 22 - protein, 25 aa, Homo sapiens
YATILEMQAMMTLDPQDILLAGNMM

SEQ ID NO: 23 - protein, 25 aa, Homo sapiens
SWIHCWKYLSVQSQLFRGSSLLFRR

SEQ ID NO: 24 - protein, 25 aa, Homo sapiens
YDNKGITYLFDLYYESDEFTVDAAR

SEQ ID NO: 25 - protein, 25 aa, Homo sapiens
AQAAKNKGNKYFQAGKYEQAIQCYT

SEQ ID NO: 26 - protein, 25 aa, Homo sapiens
QPMLPIGLSDIPDEAMVKLYCPKCM

SEQ ID NO: 27 - protein, 23 aa, Homo sapiens
HRGAIYGSSWKYFTFSGYLLYQD

SEQ ID NO: 28 - protein, 25 aa, Homo sapiens
VIQTSKYYMRDVIAIESAWLLELAP

SEQ ID NO: 29 - protein, 25 aa, Homo sapiens
PRGVDLYLRILMPIDSELVDRDVVH

SEQ ID NO: 30 - protein, 25 aa, Homo sapiens
QIEQDALCPQDTYCDLKSRAEVNGA

SEQ ID NO: 31 - protein, 25 aa, Homo sapiens
ALASAILSDPESYIKKLKELRSMLM

SEQ ID NO: 32 - protein, 137 aa, Homo sapiens
MLLPFYPPDEALEIGLELNSSALPPTILPQAPSGPSYATYLQPAQAQMLTPKPLRRNNSYTSYIMAICGMPLDSFRVIQTSKYYMRDVIAIESAWLLELAPHIHRAGGLFVADAIQVGFGRIGKHFGYPYDVPDYAS

SEQ ID NO: 33 - protein, 368 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMLLPFYPPDEALEIGLELNSSALPPTILPQAPSGPSYATYLQPAQAQMLTPKPLRRNNSYTSYIMAICGMPLDSFRVIQTSKYYMRDVIAIESAWLLELAPHIHRAGGLFVADAIQVGFGRIGKHFGYPYDVPDYAS

SEQ ID NO: 34 - protein, 165 aa, Homo sapiens
MDAMKRGLCCVLLLCGAVFVSPSQEIHARLLPFYPPDEALEIGLELNSSALPPTILPQAPSGPSYATYLQPAQAQMLTPKPLRRNNSYTSYIMAICGMPLDSFRVIQTSKYYMRDVIAIESAWLLELAPHIHRAGGLFVADAIQVGFGRIGKHFGYPYDVPDYAS

SEQ ID NO: 35 - protein, 807 aa, Homo sapiens
MDAMKRGLCCVLLLCGAVFVSPSQEIHARPGPQNFPPQNMFEFPPHLSPPLLPPGAQEEPQVEPLDFSLPKQQGELLERAVFAGSDDPFATPLSMSEMDRRNDAHSGQNHLKEMAISVLEARACAAAGQILPQAPSGPSYATYLQPAQAQMLTPMSYAEKSDEITKDEWMEKLGAGKGKYYAVNFSMRDGIDDESYGQYRGADKLCRKASSVKLVKTSPELSEDSNLQARLTSYETLKKSLSKIREESHSFIHAAMGMAVTWCAAIMTKGQYSLRTAAYVNAIEKIFKVYNEAGVTFTFEGSLAKNLSLNFQAVKENLYYEVGDPRAAYFRQAENDMYIRMALLATVLLRSQMVMKMREYFCNLHGFVDIETPDLLAFERKLDQTVMRKRLDIQEALKIKREKCWKDATYPESFHTLESVPATGRSSQVYFTINVNLDLSEAAVVTFSKPLRRNNSYTSYIMAICGMPLDSFRTTCLAVGGLDVKFQEAALRAAPDILIYEFDYHLYGQNITMIMTSVSGHLLPDSFSIPYLTALDDLLGTALLALSFYATILEMQAMMTLDPQDILLAGNMMSWIHCWKYLSVQSQLFRGSSLLFRRYDNKGITYLFDLYYESDEFTVDAARAQAAKNKGNKYFQAGKYEQAIQCYTQPMLPIGLSDIPDEAMVKLYCPKCMHRGAIYGSSWKYFTFSGYLLYQDVIQTSKYYMRDVIAIESAWLLELAPPRGVDLYLRILMPIDSELVDRDVVHQIEQDALCPQDTYCDLKSRAEVNGAALASAILSDPESYIKKLKELRSMLMGYPYDVPDYAS

SEQ ID NO: 36 - protein, 232 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 37 - protein, 215 aa, Homo sapiens
MDDQRDLISNHEQLPILGNRPREPERCSRGALYTGVSVLVALLLAGQATTAYFLYQQQGRLDKLTITSQNLQLESLRMKLPKSAKPVSQMRMATPLLMRPMSMDNMLLGPVKNVTKYGNMTQDHVMHLLTRSGPLEYPQLKGTFPENLKHLKNSMDGVNWKIFESWMKQWLLFEMSKNSLEEKKPTEAPPKEPLDMEDLSSGLGVTRQELGQVTL

SEQ ID NO: 38 - protein, 281 aa, Homo sapiens
MADSAEDAPMARGSLAGSDEALILPAGPTGGSNSRALKVAGLTTLTCLLLASQVFTAYMVFGQKEQIHTLQKNSERMSKQLTRSSQAVAPMKMHMPMNSLPLLMDFTPNEDSKTPLTKLQDTAVVSVEKQLKDLMQDSQLPQFNETFLANLQGLKQQMNESEWKSFESWMRYWLIFQMAQQKPVPPTADPASLIKTKCQMESAPGVSKIGSYKPQCDEQGRYKPMQCWHATGFCWCVDETGAVIEGTTMRGRPDCQRRALAPRRMAFAPSLMQKTISIDDQ

SEQ ID NO: 39 - protein, 27 aa, Homo sapiens
GQKEQIHTLQKNSERMSKQLTRSSQAV

SEQ ID NO: 40 - protein, 16 aa, Homo sapiens
QIHTLQKNSERMSKQL

SEQ ID NO: 41 - protein, 11 aa, Influenza virus
GYPYDVPDYAS

SEQ ID NO: 42 - protein, 29 aa, Homo sapiens
MDAMKRGLCCVLLLCGAVFVSPSQEIHAR

SEQ ID NO: 43 - protein, 25 aa, Homo sapiens
LLPFYPPDEALEIGLELNSSALPPT

SEQ ID NO: 44 - protein, 25 aa, Homo sapiens
HIHRAGGLFVADAIQVGFGRIGKHF

(19) United States
(12) Patent Application Publication
(10) Pub. No.: US 2020/0230220 A1
(43) Pub. Date: Jul. 23, 2020

(54) NEOANTIGEN VACCINE COMPOSITION FOR TREATMENT OF CANCER

(71) Applicant: NOUSCOM AG, Basel (CH)

(72) Inventors: Alfredo NICOSIA, Naples (IT); Elisa SCARSELLI, Rome (IT); Anna Morena D' ALISE, Somma Vesuviana (IT)

(21) Appl. No.: 16/626,750

(22) PCT Filed: Jul. 12, 2018
(86) PCT No.: PCT/EP2018/069047

§ 371 Date: Dec. 26, 2019

(30) Foreign Application Priority Data
Jul. 12, 2017 (EP) 17181026.0
Nov. 3, 2017 (EP) 17200036.6

(51) Int. Cl. A61K 39/00; C12N 15/86; C12N 7/00

(52) U.S. Cl. CPC A61K 39/0011; C12N 15/86; A61K 2039/53; C12N 2710/10043; A61K 2039/5256; C12N 7/00

(57) ABSTRACT

The present invention provides a polypeptide comprising at least four different tumor-specific neo-antigens fused to, at least one T cell enhancer amino acid sequence, a nucleic acid sequence encoding such polypeptide, a vector comprising such nucleic acid sequence and a collection of vectors comprising such vectors. Further provided are compositions of matter comprising in admixture or separately a vaccine comprising the polypeptide, the nucleic acid sequence the vector or the collection of vectors of the invention and at least one modulator of a checkpoint molecule or another type of immunomodulator for use in treating cancer.

The present invention provides a polypeptide comprising at least four different tumor-specific neo-antigens fused to, at least one T cell enhancer amino acid sequence, a nucleic acid sequence encoding such polypeptide, a vector comprising such nucleic acid sequence and a collection of vectors comprising such vectors. Further provided are compositions of matter comprising in admixture or separately a vaccine comprising the polypeptide, the nucleic acid sequence the vector or the collection of vectors of the invention and at least one modulator of a checkpoint molecule or another type of immunomodulator for use in treating cancer.

### BACKGROUND OF THE INVENTION

Very recently, new modalities for the treatment of cancer have been developed. Studies on the interaction between the immune system and tumour identified key pathways for evading host immune responses allowing the development of immune checkpoint inhibitors (CPIs) antibodies to unleash the power of the T-cell anti-tumour activity. Despite their success, checkpoint inhibitors are effective in a minority of treated patients. Analysis of the patterns of T cell immune response during CPIs treatment showed that a very limited number of T cell specificities against the tumor cells can be reactivated during the CPI treatment (Alsaab, H. O., et al. (2017) Front Pharmacol, 8: p. 561).

Several tumor antigens have been identified and classified in different categories: cancer-germ-line, tissue differentiation antigens and neo-antigens derived from mutated self-proteins

Fritsch, E. F., et al. (2014) Cancer Immunol Res. 2 (6): 522-9. The contribution of the immune responses against self-antigens during treatment with CPI is still a matter of debate (reviewed in Fritsch, E. F., et al. (2014) supra). A particular and preferred category of cancer antigens that has been shown to be reactivated during CPI treatment are neo-antigens. Recently, compelling evidences support the notion that neo-antigens, generated in the tumor as a consequence of mutations in coding sequences of expressed genes, represent a promising target for vaccination against cancer (Kandoth, C., et al. (2013) Nature 502 (7471): 333). Mutated proteins derived from genetic changes in coding regions of the genome can form cancer-specific neo-antigens. Cancer neo-antigens are antigens present exclusively on tumor cells and not on normal cells. Neo-antigens are generated by DNA mutations in tumor cells and have been shown to play a significant role in recognition and killing of tumor cells by the T cell mediated immune response.

The advent of next generation sequencing (NGS), which allows to determine the complete sequence of a cancer genome, in a timely and inexpensive manner, unveiled the mutational spectra of human tumors (Ott, P. A., et al. (2017) Nature 547 (7662): 217). The most frequent type of mutation is a non-synonymous single nucleotide variant (SNV) and the median number of single nucleotide variants found in tumors varies considerably according to their histology. Some tumours, like NSCLC and melanoma, have a high mutational burden and a median number of mutations above 200, with some outliers having more than 1000 mutations.

Recently two different personalized vaccination approaches based either on RNA or on peptides have been evaluated in phase-I clinical studies. The data obtained shows that vaccination can both expand the limited number of pre-existing neoantigen-specific T cells and induce a broader repertoire of new T-cell specificity in cancer patients (Ott, P. A., et al. (2017) supra and Sahin, U., et al. (2017) Nature 547 (7662): 222). The main limitation of both approaches is the maximum number of neoantigens that are targeted by these vaccination approaches. The upper limit for the peptide-based approach, based on the published data, is of twenty peptides and was not reached in all patients because in some cases peptides could not be synthesized. The described upper limit for the RNA-based approach is even lower, since only include 10 mutations were included in each vaccine. Clinical data showing efficacy of these vaccination approaches are not yet available. In cancer vaccination, it is important to avoid tumor escape through the emergence of tumor variants not recognized by vaccine induced T cells. The challenge for a cancer vaccine to cure cancer is to induce a seemingly diverse population of immune T cells capable of recognising and eliminating the largest number of cancer cells at once, therefore it is desirable that the vaccine encodes a quite large number of tumor antigens.

Further, WO 2017/118702 A1 discloses an example of a construct with only 10 neo-antigens connected by linkers demonstrating however immunogenicity of only a few neo-antigens and not efficacy. In fact, none of the previous studies showed efficacy in high tumor burden models.

In cancer vaccination, it is important to avoid tumor escape through the emergence of antigens not recognized by vaccine induced T cells. The challenge for a cancer vaccine to cure cancer is to induce a seemingly diverse population of immune T cells capable of recognising and eliminating the largest number of cancer cells at once, therefore it is desirable that the vaccine encodes a quite large number of tumor antigens.

We expect based on our preclinical data that a vaccine based on a limited number of neoantigens is perfectly suited as standalone treatment for the prevention of cancer or treatment of minimal residual disease, that is cancer diagnosed by molecular methods like circulating tumor cell free DNA. Minimal residual disease is often below the limit of detection using imaging methods for example Computed Tomography (CT) scans, Magnetic Resonance Imaging (MRI), isotopic diagnostics with radioactive tracers that are detected by scintigraphy in Positron Emission Tomography (PET). Clinical investigations in nuclear medicine demonstrates that minimum lesion detectability is about 1.5 cm in diameter. Moreover, we expect based on our preclinical data that only a vaccine satisfying both a) based on many neoantigens (i.e. >25) and b) combined with a immunomodulator like a checkpoint molecule inhibitor, can effectively control established tumors, meaning by established tumors, tumor masses that can be diagnosed by means of imaging.

To overcome these and further disadvantages of the prior art based on vaccines targeting a limited number of neoantigens the present invention provides a polynucleotide sequence encoding for many different tumor neo-antigens joined head to tail (i.e. 31) and fused to at least one T cell enhancer amino acid sequence, such as Tissue Plasminogen Activator (TPA) leader sequence or invariant chain, and vectors comprising these nucleic acids. If a vectored vaccine comprising such nucleic acid or is administered in combination with a modulator of a checkpoint molecule, a high treatment rate is elicited.

The present invention is based on the discovery that activation of the immune system against very weak immunogens like those present in the tumor, including most neo-antigens, requires a potent immunization platform and needs to be combined with peculiar structure of the encoded antigens.

Many neo-antigens are derived from point mutations, non-synonymous SNV, that are the most frequent type of mutations found in tumors. A single amino acid change in a protein sequence very rarely generates a novel epitope able to induce a potent immune response, in most cases this small change either does not generate a novel epitope at all or may generate a very weak one. The genetic vaccination platform based on adenovirus, in particular Great Apes derived Adenovirus (GAd) viral vector was shown to be very potent for induction of T cell responses and it is suitable for encoding large antigens in the format of artificial genes composed of polynucleotides encoding fragments from different proteins linked one after the other.

Unexpectedly, when the inventors used this platform in the context of neo-antigens, the inventors were unable to induce any immune response. The inventors identified specific sequences able to restore immunogenicity, which is referred to in the present application as “T cell enhancer amino acid sequence”, when fused to strings of cancer neo-antigens. Such T cell enhancer amino acid sequence was suitable in overcoming the lack of or poor immunogenicity of the neo-antigens. Preferably these sequences are fused upstream of the neo-antigens coding sequence. Among T cell enhancer amino acid sequences the inventors identified the Tissue plasminogen leader sequence (TPA) leader sequence and the invariant chain (INV), variants and fragments thereof showing the ability to restore immunogenicity. The inventors also discovered that neoantigens did not need to be connected by linkers to restore immunogenicity.

A relevant further aspect of this invention relates to the number of immunogenic neo-antigens needed for an effective cancer vaccination. The inventors discovered that a genetic vaccine, based on a Great Ape derived adenoviral vector, encoding a small number of neo-antigens, although very effective as stand-alone treatment in a prophylactic setting, when used in a therapeutic setting in the presence of large established tumors is not effective and does not synergize with an immunomodulatory molecule able to reverse T cell exhaustion, like an anti-PD-1 antibody. Instead, a larger vaccine construct encoding for more than thirty neo-antigens joined head to tail without linkers and fused to a T cell enhancer showed potent synergistic anti-tumor activity when administered in conjunction with an anti-PD-1 antibody.

### FIGURE LEGENDS

FIG. 1: Immunogenicity of GAd vectors encoding for human INV full length (CT26-5-INV) or TPA (CT26-5 TPA) sequence linked to CT26 pentatope antigen (CT26-5).Values reported were obtained by an ELISpot assay on spleen cells of immunized animals. Splenocytes were stimulated ex vivo three weeks post vaccination (dose of 5×10\hat{ }8vp) with a pool of five synthetic peptides corresponding to the sequences of the five mutations containing neo-antigens. Responses are expressed as number of T cells producing IFN γ per millions of splenocytes.

FIG. 2: Immunogenicity of GAd-CT26-31 TPA and GAd-CT26-5 TPA vectors. GAd vectors were injected intramuscular at dose of 5×10\hat{ }8 vp and T cell responses were measured by IFN-γ ELISpot three weeks post immunization and here expressed as number of T cells producing INF y per million of splenocytes. Responses against cancer mutations resulted to be immunogenic are shown. Neo-antigens #5, #18, #28 are shared by the two vectors. The dashed line represents a threshold for a positive response.

FIG. 3: Prophylactic vaccination with GAd-CT26-5 and GAd-CT26-31 vectors encoding CT26 neo-antigens effectively controls tumor development. Mice (n=8-10/group) were vaccinated with GAd-CT26-5 or GAd-CT26-31 and 2 weeks after immunization were injected s.c. with CT26 cells. Tumor growth was monitored over time. Tumor volume measured 28 days post inoculation in GAds versus untreated (mock) mice is shown.

FIG. 4: Early vaccination with GAd-CT26-5 and GAd-CT26-31 vectors effectively controls tumor growth. Mice (n=8-10/group) were inoculated i.v. with CT26 cells (day 0) and left untreated (Cntr) or injected with 5×10\hat{ }8 vp of GAd-CT26-5 or GAd-CT26-31 at day 3. The number of lung nodules counted at day 16 is shown.

FIG. 5: Efficacy of GAd vaccines in animals with high tumour burden requires targeting many neo-antigens and the combination with anti-PD1. Mice were inoculated s.c. with CT26 cells. One week later, mice were randomized according to tumor volume (mean of 70-100 mm³). Treatment with GAds vaccine started at day 0. A) Tumor growth in individual mice over time is shown in mice vaccinated with GAd-CT26-31 versus cntr (untreated) mice. B) Efficacy of anti-PD1 and combination of anti-PD1 with GAd-CT26-5 or GAd-CT26-31. Vaccine was administered at day 0 (im), while anti-PD1 was given twice per week until day 16 (ip). Shown is tumor growth in individual mice over time. Statistics are calculated by a Chi square test evaluating the number of cured mice (responders) versus non-responder mice.

FIG. 6: neoAg-specific T cell responses measured by IFN-γ ELISpot in tumor bearing mice receiving GAd-CT26-31 and anti-PD1 treatment (day30). Responses are measured on splenocytes stimulated in presence of synthetic peptides corresponding to the immunogenic neoAgs and expressed as number of T cells producing IFN-γ per millions of splenocytes.

FIG. 7: Tumor growth in tumor bearing mice treated with GAd-CT26-31 and anti-PD1 in the presence of a CD4+ T cell (CD4 depleted) or CD8+ T cell (CD8 depleted) depleting antibody or in an undepleted T cell control group. Data represent at least 2 independent experiments. Statistical significance is indicated by *(P<0.05 by Fisher exact test) or by NS (not significant).

FIG. 8: Significant difference (onesided Wilcoxon test) of intra-tumor TCR diversity (number of clonotypes) between mice from the combined treatment (left) and mice from only anti-PD1 treatment (right). Combined treatment responder mice (left, filled circles), combined treatment non-responder mice (left, filled boxes), anti-PD1 only treatment responder mice (right, triangles pointing upwards), anti-PD1 only treatment non-responder mice (right, triangles pointing downwards).

### SUMMARY OF THE INVENTION

In a first aspect the present invention relates to a polypeptide comprising at least 25 different tumor-specific neo-antigens and at least one T cell enhancer amino acid sequence.

In a second aspect the present invention relates to a nucleic acid encoding the polypeptide of the first aspect of the invention.

In a third aspect the present invention relates to a vector comprising the nucleic acid of the second aspect of the present invention operatively linked to an expression control sequence.

In a fourth aspect the present invention relates to a collection of one or more expression vectors each comprising a nucleic acid according to the second aspect of the invention, wherein each expression vector is selected from the group consisting of a plasmid; a cosmid; an RNA; an RNA-formulated with an adjuvant; an RNA formulated in liposomal particles; a self-amplifying RNA (SAM); a SAM formulated with an adjuvant; a SAM formulated in liposomal particles; a viral vector; preferably an alphavirus vector, a venezuelan equine encephalitis (VEE) virus vector, a sindbis (SIN) virus vector, a semliki forest virus (SFV) virus vector, a simian or human cytomegalovirus (CMV) vector, a Lymphocyte choriomeningitis virus (LCMV) vector, a retroviral or lentiviral vector. P preferably a replication competent or incompetent Great Apes derived adenoviral vector preferably derived from chimpanzee or bonobo or gorilla, a poxvirus vector, a vaccinia virus vector or a modified vaccinia ankara (MVA) vector.

In a fifth aspect the present invention relates to a composition comprising a vaccine comprising the polypeptide of the first aspect, the nucleic acid of the second aspect of the invention, the vector of claim the third aspect of the invention or a collection of vectors according to the fourth aspect of the invention and at least one modulator of a checkpoint molecule or a nucleic acid encoding the modulator or a vector comprising the nucleic acid encoding the modulator for use in preventing or treating a proliferative disease in a subject.

In a sixth aspect the present invention relates to a vaccination kit comprising in separate packaging:
(i) a vaccine comprising the polypeptide of the first aspect of the invention, the nucleic acid of the second aspect of the invention, the vector of the third aspect of the invention or a collection of vectors according to the fourth aspect of the invention; and
(ii) at least one modulator of a checkpoint molecule or a nucleic acid encoding the modulator or a vector comprising the nucleic acid encoding the modulator.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland) and as described in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996, and the United States Pharmacopeia-25/National Formulary-20, published by the United States Pharmcopeial Convention, Inc., Rockville Md., 2001.

Throughout this specification and the claims which follow, unless the context requires otherwise, the word “comprise”, and variations such as “comprises” and “comprising”, will be understood to imply the inclusion of a stated feature, integer or step or group of features, integers or steps but not the exclusion of any other feature, integer or step or group of integers or steps. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, some definitions of terms frequently used in this specification are provided. These terms will, in each instance of its use, in the remainder of the specification have the respectively defined meaning and preferred meanings.

The terms “polynucleotide” and “nucleic acid” are used interchangeably herein and are understood as a polymeric or oligomeric macromolecule made from nucleotide monomers. Nucleotide monomers are composed of a nucleobase, a five-carbon sugar (such as but not limited to ribose or 2′-deoxyribose), and one to three phosphate groups. Typically, a nucleic acid is formed through phosphodiester bonds between the individual nucleotide monomers. In the context of the present invention preferred nucleic acid molecules include but are not limited to ribonucleic acid (RNA), modified RNA, deoxyribonucleic acid (DNA), and mixtures thereof such as e.g. RNA-DNA hybrids. The nucleic acids, can e.g. be synthesized chemically, e.g. in accordance with the phosphotriester method (see, for example, Uhlmann, E. & Peyman, A. (1990) Chemical Reviews, 90, 543-584).

As used herein, the term “protein”, “peptide”, “polypeptide”, “peptides” and “polypeptides” are used interchangeably throughout. These terms are used in the context of the present invention to refer to both naturally occurring peptides, e.g. naturally occurring proteins and synthesized peptides that may include naturally or non-naturally occurring amino acids.

The term “neo-antigen” is used in the context of the present invention to refer to an antigen not present in normal/germline cells but which occurs in transformed, in particular cancerous cells. A neo-antigen may comprise one or more, e.g. 2, 3, 4, 5 or more neo-epitopes. It is preferred that the length of each neo-antigen included in the polypeptide of the present invention is selected in such to ascertain that they there is a low likelihood of comprising epitopes that occur in normal/germline cells. Typically, this can be ascertained that the neo-antigen comprises 12 or less amino acids C-terminally and/or N-terminally of the amino acid change(s) that created a neo-epitope.
The mutated cancer protein is generated by a mutation occurring at level of DNA and wherein the mutated protein can comprise
a) one or more single aa changes caused by a point mutation non-synonymous SNV; and/or
b) a non-wildtype amino acid sequence caused by insertions/deletions resulting in frame shifted peptide; and/or
c) a non-wildtype amino acid sequence caused by alteration of exon boundaries or by mutations generating intron retention; and/or
d) a mutated cancer protein generated by a gene fusion event.

A neo-antigen that is the result of one or more single amino acid changes caused by a genomic point mutation non-synonymous SNV is referred to in the context of the present invention as a single amino acid mutant peptide.

The term “frame-shift peptide” is used in the context of the present invention to refer to the complete non wild-type translation product of the protein-encoding segment of a nucleic acid comprising an insertion or deletion mutations causing a shift of the Open Reading Frame (ORF).

The term “open reading frame” abbreviated “ORF” is used in the context of the present invention to refer to a sequence of nucleotides that can be translated into a consecutive string of amino acids. Typically, an ORF contains a start codon, a subsequent region usually having a length which is a multiple of 3 nucleotides, but does not contain a stop codon (TAG, TAA, TGA, UAG, UAA, or UGA) in the given reading frame. An ORF codes for a protein where the amino acids into which it can be translated form a peptide-linked chain.

A neo-antigen that is the result of a non-wildtype amino acid sequence caused by alteration of exon boundaries or by mutations generating intron retention is referred to in the context of the present invention as a splice site mutant peptide.

A neo-antigen that is the result of a mutated cancer protein generated by a gene fusion event is referred to in the context of the present invention as a read-through mutation peptide.

The term “expression cassette” is used in the context of the present invention to refer to a nucleic acid molecule which comprises at least one nucleic acid sequence that is to be expressed, e.g. a nucleic acid encoding the string of neo-antigens fused to invariant chain of the present invention or a part thereof, operably linked to transcription and translation control sequences. Preferably, an expression cassette includes cis-regulating elements for efficient expression of a given gene, such as promoter, initiation-site and/or polyadenylation-site. Preferably, an expression cassette contains all the additional elements required for the expression of the nucleic acid in the cell of a patient. A typical expression cassette thus contains a promoter operatively linked to the nucleic acid sequence to be expressed and signals required for efficient polyadenylation of the transcript, ribosome binding sites, and translation termination. Additional elements of the cassette may include, for example enhancers. An expression cassette preferably also contains a transcription termination region downstream of the structural gene to provide for efficient termination. The termination region may be obtained from the same gene as the promoter sequence or may be obtained from a different gene.

The term “operably linked” as used in the context of the present invention refers to an arrangement of elements, wherein the components so described are configured so as to perform their usual function. A nucleic acid is “operably linked” when it is placed into a functional relationship with another nucleic acid sequence. For example, a promoter is operably linked to one or more transgenes, if it affects the transcription of the one or more transgenes. Further, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The terms “vector” or “expression vector” are used interchangeably and refer to a polynucleotide or a mixture of a polynucleotide and proteins capable of being introduced or of introducing the collection of nucleic acids of the present invention or one nucleic acid that is part of the collection of nucleic acids of the invention into a cell, preferably a mammalian cell. Examples of vectors include but are not limited to plasmids, cosmids, phages, viruses or artificial chromosomes. In particular, a vector is used to transport the promoter and the collection of the nucleic acids or one nucleic acid that is part of the collection of nucleic acids of the invention into a suitable host cell. Expression vectors may contain “replicon” polynucleotide sequences that facilitate the autonomous replication of the expression vector in a host cell. Once in the host cell, the expression vector may replicate independently of or coincidental with the host chromosomal DNA, and several copies of the vector and its inserted DNA can be generated. In case that replication incompetent expression vectors are used—which is often the case for safety reasons—the vector may not replicate but merely direct expression of the nucleic acid. Depending on the type of expression vector the expression vector may be lost from the cell, i.e only transiently expresses the neo-antigens encoded by the nucleic acid or may be stable in the cell. Expression vectors typically contain expression cassettes, i.e. the necessary elements that permit transcription of the nucleic acid into an mRNA molecule.

The term “expression control sequence” refers to a tag suitable for determining or measuring the expression. Suitable tags are known in the art. In the context of the present invention, suitable tags can be protein tags whose peptide sequences are linked to the polypeptide of the invention. Protein tags may e.g. encompass affinity tags, solubilization tags, chromatography tags, epitope tags, or Fluorescence tags. Affinity tags are appended to proteins so that they can be purified from their crude biological source using an affinity technique. These include chitin binding protein (CBP), maltose binding protein (MBP), and glutathione-S-transferase (GST). The poly(His) tag is a widely used protein tag which binds to metal matrices. Solubilization tags are used, especially for recombinant proteins expressed in chaperone-deficient species to assist in the proper folding in proteins and keep them from precipitating. These include thioredoxin (TRX) and poly(NANP). Some affinity tags have a dual role as a solubilization agent, such as MBP, and GST. Chromatography tags are used to alter chromatographic properties of the protein to afford different resolution across a particular separation technique. Often, these consist of polyanionic amino acids, such as FLAG-tag. Epitope tags are short peptide sequences which are chosen because high-affinity antibodies can be reliably produced in many different species. These are usually derived from viral genes, which explain their high immunoreactivity. Epitope tags include V5-tag, Myc-tag, and HA-tag. These tags are particularly useful for western blotting, immunofluorescence and immunoprecipitation experiments, although they also find use in antibody purification. Fluorescence tags are used to give visual readout on a protein. GFP and its variants are the most commonly used fluorescence tags. More advanced applications of GFP include using it as a folding reporter (fluorescent if folded, colorless if not). Further examples of fluorophores include fluorescein, rhodamine, and sulfoindocyanine dye Cy5.

Examples of such tag include but are not limited to AviTag, Calmodulin-tag, polyglutamate tag, E-tag, FLAG-tag, HA-tag, His-tag, Myc-tag, S-tag, SBP-tag, Softag 1, Softag 3, Strep-tag, TC tag, V5 tag, VSV-tag, Xpress tag, Isopeptag, SpyTag, BCCP tag, Glutathione-S-transferase-tag, Green fluorescent protein-tag, Maltose binding protein-tag, Nus-tag, Thioredoxin-tag, Fc-tag, and Ty tag. Most preferred is the HA tag (HA peptide sequence according to SEQ ID NO: 41).

The term “antigen” is used in the context of the present invention to refer to any structure recognized by molecules of the immune response, e.g. antibodies, T cell receptors (TCRs) and the like. Preferred antigens are cellular proteins that are associated with a particular disease. Antigens are recognized by highly variable antigen receptors (B-cell receptor or T-cell receptor) of the adaptive immune system and may elicit a humoral or cellular immune response. Antigens that elicit such a response are also referred to as immunogen. A fraction of the proteins inside cells, irrespective of whether they are foreign or cellular, are processed into smaller peptides and presented to by the major histocompatibility complex (MHC).

The term “epitope”, also known as antigenic determinant, is used in the context of the present invention to refer to the segment of an antigen, preferably peptide that is bound by molecules of the immune system, e.g. B-cell receptors, T-cell receptors or antibodies. The epitopes bound by antibodies or B cells are referred to as “B cell epitopes” and the epitopes bound by T cells are referred to as “T cell epitopes”. In this context, the term “binding” preferably relates to a specific binding, which is defined as a binding with an association constant between the antibody or T cell receptor (TCR) and the respective epitope of 1×10⁵ M-1 or higher, preferably of 1×10⁶ M-1, 1×10⁷M-1, 1×10⁸M-1 or higher. The skilled person is well aware how to determine the association constant (see e.g. Caoili, S. E. (2012) Advances in Bioinformatics Vol. 2012). Preferably, the specific binding of antibodies to an epitope is mediated by the Fab (fragment, antigen binding) region of the antibody, specific binding of a B-cell is mediated by the Fab region of the antibody comprised by the B-cell receptor and specific binding of a T-cell is mediated by the variable (V) region of the T-cell receptor. T cell epitopes are presented on the surface of an antigen presenting cell, where they are bound to Major Histocompatiblilty (MHC) molecules. There are at least two different classes of MHC molecules termed MHC class I, II respectively. Epitopes presented through the MHC-I pathway elicit a response by cytotoxic T lymphocytes (CD8+ cells), while epitopes presented through the MHC-II pathway elicit a response by T-helper cells (CD4+ cells). T cell epitopes presented by MHC Class I molecules are typically peptides between 8 and 11 amino acids in length and T cell epitopes presented by MHC Class II molecules are typically peptides between 13 and 17 amino acids in length. MHC Class III molecules also present non-peptidic epitopes as glycolipids. Accordingly, the term “T cell epitope” preferably refers to a 8 to 11 or 13 to 17 amino acid long peptide that can be presented by either a MHC Class I or MHC Class II molecule. Epitopes usually consist of chemically active surface groupings of amino acids, which may or may not carry sugar side chains and usually have specific three-dimensional structural characteristics, as well as specific charge characteristics. Conformational and non-conformational epitopes are distinguished in that the binding to the former but not the latter is lost in the presence of denaturing solvents.

The term “T cell enhancer amino acid sequence” refers to a polypeptide sequences that when fused to an antigenic sequence increases the induction of T cells against neo-antigens in the context of a genetic vaccination. Examples of T cell enhancers are an invariant chain sequence or fragment thereof; a tissue-type plasminogen activator leader sequence optionally including six additional downstream amino acid residues; a PEST sequence; a cyclin destruction box; an ubiquitination signal; a SUMOylation signal.

The terms “preparation” and “composition” as used in the context of the present invention are intended to include the formulation of the active compound, e.g. the Great Apes Adenovector of the present invention with a carrier and/or excipient.

“Pharmaceutically acceptable” as used in the context of the present invention means approved by a regulatory agency of the Federal or a state government or listed in the U.S. Pharmacopeia or other generally recognized pharmacopeia for use in animals, and more particularly in humans.

The term “carrier”, as used herein, refers to a pharmacologically inactive substance such as but not limited to a diluent, excipient, surfactants, stabilizers, physiological buffer solutions or vehicles with which the therapeutically active ingredient is administered. Such pharmaceutical carriers can be liquid or solid. Liquid carrier include but are not limited to sterile liquids, such as saline solutions in water and oils, including but not limited to those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. Examples of suitable pharmaceutical carriers are described in “Remington's Pharmaceutical Sciences” by E. W. Martin.

Suitable pharmaceutical “excipients” include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like.

“Surfactants” include anionic, cationic, and non-ionic surfactants such as but not limited to sodium deoxycholate, sodium dodecylsulfate, Triton X-100, and polysorbates such as polysorbate 20, polysorbate 40, polysorbate 60, polysorbate 65 and polysorbate 80.

“Stabilizers” include but are not limited to mannitol, sucrose, trehalose, albumin, as well as protease and/or nuclease antagonists.

“Physiological buffer solution” that may be used in the context of the present invention include but are not limited to sodium chloride solution, demineralized water, as well as suitable organic or inorganic buffer solutions such as but not limited to phosphate buffer, citrate buffer, tris buffer (tris(hydroxymethyl)aminomethane), HEPES buffer ([4 (2 hydroxyethyl)piperazino]ethanesulphonic acid) or MOPS buffer (3 morpholino-1 propanesulphonic acid). The choice of the respective buffer in general depends on the desired buffer molarity. Phosphate buffer are suitable, for example, for injection and infusion solutions.

An “effective amount” or “therapeutically effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

As used herein, “treat”, “treating”, “treatment” or “therapy” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in an individual that has previously had the disorder(s); and (e) limiting or preventing recurrence of symptoms in individuals that were previously symptomatic for the disorder(s).

### Aspects of the Invention and Preferred Embodiments

In a first aspect the present invention relates to a polypeptide comprising at least four different tumor-specific neo-antigens and at least one T cell enhancer amino acid sequence.

The present inventors have surprisingly found that the efficacy of the treatment in a therapeutic setting with large established tumors is dependent on the number of immunogenic neo-antigens eliciting T cell responses. This is particularly evident in the context of co-administration of a modulator of a checkpoint molecule. If the number of immunogenic neo-antigens is raised beyond 3 then the treatment outcome dramatically improves. “Immunogenic” in this context means capable of eliciting a T cell response in the patient. Therefore, it is generally preferred that at least 4, at least 5, at least 6, at least 7, at least 8, at least 8, at least 9 or at least 10 of the neo-antigens are immunogenic (elicit a T cell response in a patient). The skilled person is well aware of how to measure a T cell response in a patient. One possible way is outlined in below example section.

To consistently achieve this minimal number of immunogenic neo-antigens, it is particularly preferred that the polypeptide of the first aspect comprises at least 25 tumor-specific neo-antigens, preferably at least 26, 27, 28, 29 or 30 tumor-specific neo-antigens, most preferably at least 31. While the examples section herein shows the use of 31 tumor-specific neo-antigens, it is of course possible and within the scope of the invention to increase the number further, e.g. to at least 35, at least 40, at least 45, or at least 50 tumor-specific neo-antigens. Preferably, the polypeptide comprises between (and including) 25 to 200, more preferably 25 to 150, even more preferably 25 to 100, or most preferably 25 to 80 tumor-specific neo-antigens. More preferably, the polypeptide comprises between (and including) 31 to 200, more preferably 31 to 150, even more preferably 31 to 100, or most preferably 31 to 80 tumor-specific neo-antigens. Generally, with respect to any minimum number referred to herein, it is preferred that the upper limit of tumor-specific neo-antigens is 80. This is not because it is not feasible to include more than 80, but for the purpose of being able prepare a vaccine more quickly.

It is preferred that of the at least 25 tumor-specific neo-antigens, at least 4, at least 5, at least 6, at least 7, at least 8, at least 9 or at least 10 (with increasing preference) of the neo-antigens are immunogenic. It is preferred that of the at least 26 tumor-specific neo-antigens, at least 4, at least 5, at least 6, at least 7, at least 8, at least 9 or at least 10 (with increasing preference) of the neo-antigens are immunogenic. It is preferred that of the at least 27 tumor-specific neo-antigens, at least 4, at least 5, at least 6, at least 7, at least 8, at least 9 or at least 10 (with increasing preference) of the neo-antigens are immunogenic. It is preferred that of the at least 28 tumor-specific neo-antigens, at least 4, at least 5, at least 6, at least 7, at least 8, at least 9 or at least 10 (with increasing preference) of the neo-antigens are immunogenic. It is preferred that of the at least 29 tumor-specific neo-antigens, at least 4, at least 5, at least 6, at least 7, at least 8, at least 9 or at least 10 (with increasing preference) of the neo-antigens are immunogenic. It is preferred that of the at least 30 tumor-specific neo-antigens, at least 4, at least 5, at least 6, at least 7, at least 8, at least 9 or at least 10 (with increasing preference) of the neo-antigens are immunogenic. It is preferred that of the at least 31 tumor-specific neo-antigens, at least 4, at least 5, at least 6, at least 7, at least 8, at least 9 or at least 10 (with increasing preference) of the neo-antigens are immunogenic. It is preferred that of the at least 35 tumor-specific neo-antigens, at least 4, at least 5, at least 6, at least 7, at least 8, at least 9 or at least 10 (with increasing preference) of the neo-antigens are immunogenic. It is preferred that of the at least 40 tumor-specific neo-antigens, at least 4, at least 5, at least 6, at least 7, at least 8, at least 9 or at least 10 (with increasing preference) of the neo-antigens are immunogenic. It is preferred that of the at least 45 tumor-specific neo-antigens, at least 4, at least 5, at least 6, at least 7, at least 8, at least 9 or at least 10 (with increasing preference) of the neo-antigens are immunogenic. It is preferred that of the at least 50 tumor-specific neo-antigens, at least 4, at least 5, at least 6, at least 7, at least 8, at least 9 or at least 10 (with increasing preference) of the neo-antigens are immunogenic. Furthermore, It is preferred that of the at least 25 to 200 tumor-specific neo-antigens, at least 4, at least 5, at least 6, at least 7, at least 8, at least 9 or at least 10 (with increasing preference) of the neo-antigens are immunogenic. It is preferred that of the at least 25 to 150 tumor-specific neo-antigens, at least 4, at least 5, at least 6, at least 7, at least 8, at least 9 or at least 10 (with increasing preference) of the neo-antigens are immunogenic. It is preferred that of the at least 25 to 100 tumor-specific neo-antigens, at least 4, at least 5, at least 6, at least 7, at least 8, at least 9 or at least 10 (with increasing preference) of the neo-antigens are immunogenic. It is preferred that of the at least 25 to 80 tumor-specific neo-antigens, at least 4, at least 5, at least 6, at least 7, at least 8, at least 9 or at least 10 (with increasing preference) of the neo-antigens are immunogenic. It is preferred that of the at least 31 to 200 tumor-specific neo-antigens, at least 4, at least 5, at least 6, at least 7, at least 8, at least 9 or at least 10 (with increasing preference) of the neo-antigens are immunogenic. It is preferred that of the at least 31 to 150 tumor-specific neo-antigens, at least 4, at least 5, at least 6, at least 7, at least 8, at least 9 or at least 10 (with increasing preference) of the neo-antigens are immunogenic. It is preferred that of the at least 31 to 100 tumor-specific neo-antigens, at least 4, at least 5, at least 6, at least 7, at least 8, at least 9 or at least 10 (with increasing preference) of the neo-antigens are immunogenic. It is preferred that of the at least 31 to 80 tumor-specific neo-antigens, at least 4, at least 5, at least 6, at least 7, at least 8, at least 9 or at least 10 (with increasing preference) of the neo-antigens are immunogenic.

It is also generally preferred that the tumor is at least of stage Tis or T1 (excluding Tx and T0), preferably of at least stage T2, T3 or T4. It may at the same time be of all stages N (e.g. Nx or N0) and M (e.g. M0), and in a preferred embodiment at least of stage N1, N2 or N3 and/or M1). This refers to the TNM classification, which defines the tumor stages as follows:
T: size or direct extent of the primary tumour

Tx: tumour cannot be assessed

Tis: carcinoma in situ

T0: no evidence of tumour

T1, T2, T3, T4: evidence of primary tumor, size and/or extension increasing with stage N: degree of spread to regional lymph nodes

Nx: lymph nodes cannot be assessed

N0: no regional lymph nodes metastasis

N1: regional lymph node metastasis present; at some sites, tumour spread to closest or small number of regional lymph nodes

N2: tumour spread to an extent between N1 and N3 (N2 is not used at all sites)

N3: tumour spread to more distant or numerous regional lymph nodes (N3 is not used at all sites)
M: presence of distant metastasis

M0: no distant metastasis

M1: metastasis to distant organs (beyond regional lymph nodes)

Exemplary stages envisaged to benefit in particular from the invention are Tis and any of N (preferably N1 or N2 or N3) and any of M (preferably M1), T1 and any of N (preferably N1 or N2 or N3) and any of M (preferably M1), T2 and any of N (preferably N1 or N2 or N3) and any of M (preferably M1), T3 and any of N (preferably N1 or N2 or N3) and any of M (preferably M1), and T4 and any of N (preferably N1 or N2 or N3) and any of M (preferably M1).The presence of a tumor and its spread in a patient can be detected using imaging methods, for example Computed Tomography (CT) scans, Magnetic Resonance Imaging (MRI), isotopic diagnostics with radioactive tracers that are detected by scintigraphy in Positron Emission Tomography (PET) or a combination thereof. Imaging methods can also be combined with other methods like for example ultra sound examination, endoscopic examination, mammography, biomarker detection in the blood, fine needle biopsy or a combination thereof. The size of tumors that can be detected by imaging methods depends on the method used and is about 1.5 cm in diameter for isotope imaging, about 3 mm in diameter for CT and MRI and about 7 mm in diameter for PET-based methods (Erdi. (2012) Molecular Imaging and Radionuclide Therapy 21 (1): 23).

Preferably, the the presence of a tumor (“evidence”) determined with a method selected from the group consisting of detection of circulating tumor cell free DNA, Computed Tomography (CT) scan, Magnetic Resonance Imaging (MRI), isotopic diagnostics with radioactive tracers that are detected by scintigraphy in Positron Emission Tomography (PET), and any combination of the foregoing. In one embodiment, one or more of the foregoing methods or combination thereof is a combined with a method of the group consisting of ultra sound examination, endoscopic examination, mammography, biomarker detection in the blood, fine needle biopsy and any combination of the foregoing.

In a preferred embodiment, the tumor is characterized by a lesion of at least about 3 mm in diameter, preferably at least 7 mm in diameter, and more preferably at least 1.5 cm in diameter.

Preferably, the tumor specific neo-antigen is independently selected from the group consisting of a single amino acid mutant peptide, a frame-shift peptide, a read-through mutation peptide and a splice site mutant peptide.

In a preferred embodiment of the first aspect the polypeptide comprises at least five protein fragments containing tumor-specific neo-antigens. It is preferred that the polypeptide comprises at least ten protein fragments containing tumor-specific neo-antigens. It is also preferred that the polypeptide comprises at least fifteen protein fragments containing tumor-specific neo-antigens. It is also preferred that the polypeptide comprises at least twenty protein fragments containing tumor-specific neo-antigens. It is also preferred that the polypeptide comprises at least twenty five protein fragments containing tumor-specific neo-antigens. More preferably the polypeptide comprises at least thirty protein fragments containing tumor-specific neo-antigens.

In another embodiment of the first aspect of the present invention the polypeptide comprises at least five, at least ten, at least fifteen, at least twenty, and preferably at least 30, at least 35, at least 40, at least 45, at least 50 or more tumor-specific neo-antigens. Preferably, the polypeptide comprises between 5 to 200, more preferably 15 to 150, even more preferably 25 to 100 or more preferably 30 to 50 tumor-specific neo-antigens.

In another embodiment of the first aspect of the present invention the tumor-specific neo-antigens independently of each other have a length of 8 to 50 amino acids. It is preferred that the tumor-specific neo-antigens independently of each other have a length of 9 to 45 amino acids. It is more preferred that the tumor-specific neo-antigens independently of each other have a length of 10 to 40 amino acids. It is also preferred that the tumor-specific neo-antigens independently of each other have a length of 15 to 35 amino acids. It is also preferred that the tumor-specific neo-antigens independently of each other have a length of 12 to 30 amino acids. It is more preferred that the tumor-specific neo-antigens independently of each other have a length of 13 to 28 amino acids. It is more preferred that the tumor-specific neo-antigens independently of each other have a length of 14 to 45 amino acids. It is even more preferred that the tumor-specific neo-antigens independently of each other have a length of 15 to 35 amino acids. Most preferably, the tumor-specific neo-antigens independently of each other have a length of 25 amino acids.

In another embodiment of the first aspect of the present invention each tumor-specific neo-antigens independently of each other have a length of 8 to 50 amino acids, preferably a length of 15 to 35, more preferably of 25 amino acids.

Preferably, the polypeptide comprises between 5 to 200 tumor-specific neo-antigens of a length of 8 to 50 amino acids, preferably a length of 15 to 35, more preferably of 25 amino acids; more preferably 15 to 150 tumor-specific neo-antigens of a length of 8 to 50 amino acids, preferably a length of 15 to 35, more preferably of 25 amino acids; even more preferably 25 to 100 tumor-specific neo-antigens of a length of 8 to 50 amino acids, preferably a length of 15 to 35, more preferably of 25 amino acids; or more preferably 30 to 50 tumor-specific neo-antigens tumor-specific neo-antigens of a length of 8 to 50 amino acids, preferably a length of 15 to 35, more preferably of 25 amino acids.

The overall the length of the neo-antigens within the peptide is preferably in the range of 100 to 2000 amino acids. More preferably 500 to 1000 amino acids.

In another embodiment of the first aspect of the present invention each tumor-specific neo-antigen is independently selected from the group consisting of a single amino acid mutant peptide, a frame-shift peptide, a read-through mutation peptide, and a splice site mutant peptide. Preferably, at least 80% of the tumor-specific neo-antigen are single amino acid mutant peptide, more preferably at least 85% of the tumor-specific neo-antigen are single amino acid mutant peptide, more preferably at least 90% of the tumor-specific neo-antigen are single amino acid mutant peptide and more preferably at least 95% of the tumor-specific neo-antigen are single amino acid mutant peptide.

In another preferred embodiment of the first aspect of the present invention the tumor-specific neo-antigens are linked directly to each other.

In another preferred embodiment of the first aspect of the present invention the amino acid linker sequences are included between each neo-antigen or between groups of neo-antigens. Suitable linker sequences are well known in the art and preferably comprise or consist of between 1 to 10 amino acids. Linker preferably consist or comprise small amino acids like Ser and Gly.

In another embodiment of the first aspect of the present invention amino acid linker sequences are included between each neo-antigen or between groups of neo-antigens. Preferably the linkers can derive from naturally-occurring multi-domain proteins or being generated by design. Linkers include flexible linkers and/or in vivo cleavable linkers that can be processed by cellular proteases.

In another preferred embodiment of the first aspect of the present invention the T cell enhancer amino acid sequence is selected from the group consisting of an invariant chain; a leader sequence of tissue-type plasminogen activator (TPA); a PEST sequence; a cyclin destruction box; an ubiquitination signal; a SUMOylation signal.

It is preferred that the T cell enhancer amino acid sequence is placed N-terminally within the polypeptide, more preferably at the N-terminus of the polypeptide of the present invention.

In another preferred embodiment of the first aspect of the present invention the TPA is an extended TPA leader sequence comprising the TPA leader sequence and the two to ten, preferably four to eight and more preferably the six TPA residues immediately C-terminal to the TPA leader sequence. The inventors found that having these additional residues improves the reliability of a correct cleavage of the leader sequence (correct meaning that the leader sequence is cleaved off at the same residue as in the wild-type TPA). They found that introducing only the leader sequence can lead to cleavage within the neo-antigen portion and this would cleave off a part of the neo-antigen string. It is preferred that the TPA is present at the N-terminus of the polypeptide according to the first aspect of the present invention. A preferred TPA that can be included in the polypeptide of the present invention has an amino acid sequence according to SEQ ID NO: 42.

In another preferred embodiment of the first aspect of the present invention the invariant chain is selected from the group consisting of:
(a) human invariant chain according to SEQ ID NO: 36, mouse invariant chain according to SEQ ID NO: 37, and Mandarin fish invariant chain according to SEQ ID NO: 38;
(b) an immune stimulatory fragment of an invariant chain according to (a); and/or
(c) an immune stimulatory variant of (a) or (b), wherein the variant has at least 70% sequence identity to the invariant chain according to (a) or a fragment thereof according to (b).

It is preferred that the invariant chain is a human invariant chain according to SEQ ID NO: 36. It is also preferred that the invariant chain is a mouse invariant chain according to SEQ ID NO: 37. It is also preferred that the invariant chain is a Mandarin fish invariant chain according to SEQ ID NO: 38. Such invariant chains are described in the prior art, e.g. in WO 2007/062656.

Preferably, the invariant chain is an immune stimulatory fragment of a human invariant chain according to SEQ ID NO: 36. It is further preferred that the invariant chain is an immune stimulatory fragment of a mouse invariant chain according to SEQ ID NO: 37. It is further preferred that the invariant chain is Mandarin fish invariant chain according to SEQ ID NO: 38. Such fragments have been described in the prior art, like e.g. WO 2010/057501 and WO 2015/082922. Particularly preferred fragments comprise or consist of a fragment of SEQ ID NO: 38, in particular comprising or consisting an amino acid sequence of SEQ ID NO: 39 or 40.

It is also preferred that the invariant chain is an immune stimulatory variant of a human invariant chain according to SEQ ID NO: 36 wherein the variant has at least 70% sequence identity, more preferably at least 75% sequence identity, more preferably at least 80% sequence identity, more preferably at least 85% sequence identity, more preferably at least 90% sequence identity and even more preferably at least 95% sequence identity to the invariant chain according to SEQ ID NO: 36. It is also preferred that the invariant chain is an immune stimulatory variant of a mouse invariant chain according to SEQ ID NO: 37 wherein the variant has at least 70% sequence identity, more preferably at least 75% sequence identity, more preferably at least 80% sequence identity, more preferably at least 85% sequence identity, more preferably at least 90% sequence identity and even more preferably at least 95% sequence identity to the invariant chain according to SEQ ID NO: 37. It is also preferred that the invariant chain is an immune stimulatory variant of a Mandarin fish invariant chain according to SEQ ID NO: 38 wherein the variant has at least 70% sequence identity, more preferably at least 75% sequence identity, more preferably at least 80% sequence identity, more preferably at least 85% sequence identity, more preferably at least 90% sequence identity and even more preferably at least 95% sequence identity to the invariant chain according to SEQ ID NO: 38.

The term “immune stimulatory variant” of an immune stimulatory fragment of an invariant chain means in the context of the present invention, that the activity in an assay assessing the immune stimulatory activity of neo-antigen (see, e.g. the Examples below) is at least 50% of the activity measured for the unaltered invariant or fragment thereof. Preferably at least 60%, more preferably at least 70%, more preferably at least 80%, most preferably the same or higher immune stimulatory activity.

It is further preferred that the polypeptide does not comprise a MITD (MHC class I trafficking signal) because this would direct the polypeptide into the endoplasmatic reticulum membrane after expression, which is not desirable. It is even more preferred, accordingly, that the polypeptide generally does not comprise an element directing it into the endoplasmatic reticulum membrane after expression. In a particular embodiment, the polypeptide is linked at the C-terminus to a tag (expression control sequence) as defined herein. In this embodiment it is preferred that the tag is at the C-terminus of the polypeptide (i.e. there is no further element). If the polypeptide does not comprise a tag, it is preferred that the C-terminus of the polypeptide is a neo-antigen (i.e. there is no further element that is not a neo-antigen).

In a second aspect the present invention relates to a nucleic acid encoding the polypeptide of the first aspect of the invention.

In a third aspect the present invention relates to a vector comprising the nucleic acid of the second aspect of the present invention operatively linked to an expression control sequence.

In a preferred embodiment of the collection of expression vectors of the seventh aspect each expression vector of the collection is independently selected from the group consisting of a plasmid; a cosmid; an RNA; an RNA-formulated with an adjuvant; an RNA formulated in liposomal particles; a self-amplifying RNA (SAM); a SAM formulated with an adjuvant; a SAM formulated in liposomal particles; a viral vector; preferably an alphavirus vector, a venezuelan equine encephalitis (VEE) virus vector, a sindbis (SIN) virus vector, a semliki forest virus (SFV) virus vector, also preferably a replication competent or incompetent adenoviral vector preferably derived from chimpanzee or bonobo or gorilla, a poxvirus vector, a vaccinia virus vector or a modified vaccinia ankara (MVA) vector, a simian or human cytomegalovirus (CMV) vector, a Lymphocyte choriomeningitis virus (LCMV) vector, a retroviral or lentiviral vector. It is preferred that all expression vectors used in one collection are of the same type, e.g. replication incompetent adenoviral vectors.

The most preferred expression vectors are adenoviral vectors, in particular adenoviral vectors derived from human or non-human great apes. Preferred great apes from which the adenoviruses are derived are Chimpanzee (Pan), Gorilla (Gorilla) and orangutans (Pongo), preferably Bonobo (Pan paniscus) and common Chimpanzee (Pan troglodytes). Typically, naturally occurring non-human great ape adenoviruses are isolated from stool samples of the respective great ape. The most preferred vectors are non-replicating adenoviral vectors based on hAd5, hAd11, hAd26, hAd35, hAd49, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd55, ChAd63, ChAd 73, ChAd82, ChAd83, ChAd146, ChAd147, PanAd1, PanAd2, and PanAd3 vectors or replication-competent Ad4 and Ad7 vectors. The human adenoviruses hAd4, hAd5, hAd7, hAd11, hAd26, hAd35 and hAd49 are well known in the art. Vectors based on naturally occurring ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63 and ChAd82 are described in detail in WO 2005/071093. Vectors based on naturally occurring PanAdl, PanAd2, PanAd3, ChAd55, ChAd73, ChAd83, ChAd146, and ChAd147 are described in detail in WO 2010/086189.

In a fourth aspect the present invention relates to a collection of one or more expression vectors each comprising a nucleic acid according to claim 11, wherein each expression vector is selected from the group consisting of a plasmid; a cosmid; an RNA; an RNA-formulated with an adjuvant; an RNA formulated in liposomal particles; a self-amplifying RNA (SAM); a SAM formulated with an adjuvant; a SAM formulated in liposomal particles; a viral vector; preferably an alphavirus vector, a venezuelan equine encephalitis (VEE) virus vector, a sindbis (SIN) virus vector, a semliki forest virus (SFV) virus vector, a simian or human cytomegalovirus (CMV) vector, a Lymphocyte choriomeningitis virus (LCMV) vector, a retroviral or lentiviral vector. P preferably a replication competent or incompetent Great Apes derived adenoviral vector preferably derived from chimpanzee or bonobo or gorilla, a poxvirus vector, a vaccinia virus vector or a modified vaccinia ankara (MVA) vector.

In a fifth aspect the present invention relates to a composition comprising a vaccine comprising the polypeptide of the first aspect, the nucleic acid of the second aspect of the invention, the vector of claim the third aspect of the invention or a collection of vectors according to the fourth aspect of the invention and at least one modulator of a checkpoint molecule or a nucleic acid encoding the modulator or a vector comprising the nucleic acid encoding the modulator for use in preventing or treating a proliferative disease in a subject.

In a preferred embodiment of the fifth aspect the modulator of a checkpoint molecule is selected from the group consisting of:
(a) an agonist of a tumor necrosis factor (TNF) receptor superfamily member, preferably of CD27 (e.g. Varlilumab), CD40 (e.g. CP-870,893), OX40 (e.g. INCAGN01949 or MEDI0562), GITR (e.g. MEDI1873) or CD137 (e.g. Utomilumab);
(b) an antagonist of PD-1 (e.g. an antibody such as pembrolizumab or nivolumab), PD-L1 (e.g. an antibody such as atezolizumab), CD274 (atezolizumab or Durvalumab), A2AR (e.g. Preladenant), B7-H3 (e.g. MGA271), B7-H4, BTLA, CTLA-4 (e.g. Tremelimumab or AGEN1884), IDO, KIR, LAGS, TIM-3 (e.g. CA-327 or RMT3-23), or VISTA (e.g. CA-170) or an antagonist of a B7-CD28 superfamily member, preferably of CD28 or ICOS or an antagonist of a ligand thereof.

Preferred immunomodulators are T cell growth factors like IL-2, IL-12, or IL-15.

In a preferred embodiment of the fifth aspect of the invention the administration of the modulator of a checkpoint molecule is initiated before initiation of administration of the vaccine, or wherein administration of the checkpoint inhibitor is initiated after initiation of administration of the vaccine, or wherein administration of the checkpoint inhibitor is initiated simultaneously with the initiation of administration of the vaccine.

In a preferred embodiment of the fifth aspect of the invention the vaccination regimen is a heterologous prime boost with two different viral vectors. Preferred combinations are Great Apes derived adenoviral vector for priming and a poxvirus vector, a vaccinia virus vector or a modified vaccinia ankara (MVA) vector for boosting being. Preferably these are administered sequentially with an interval of at least 1 week, preferably of 6 weeks.

In a preferred embodiment of the fifth aspect of the invention the subject is suffering from or is at risk of suffering from:
(a) Malignant neoplasms of lip, oral cavity and pharynx; and/or
(b) Malignant neoplasms of digestive organs; and/or
(c) Malignant neoplasms of respiratory and intrathoracic organs; and/or
(d) Malignant neoplasms of bone and articular cartilage; and/or
(e) Melanoma and other malignant neoplasms of skin; and/or
(f) Malignant neoplasms of mesothelial and soft tissue; and/or
(g) Malignant neoplasm of breast; and/or
(h) Malignant neoplasms of female genital organs; and/or
(i) Malignant neoplasms of male genital organs; and/or
(j) Malignant neoplasms of urinary tract; and/or
(k) Malignant neoplasms of eye, brain and other parts of central nervous system; and/or
(l) Malignant neoplasms of thyroid and other endocrine glands; and/or
(m) Malignant neoplasms of lymphoid, haematopoietic and related tissue.

Generally, it is preferred that the subject has a tumor at a TNM stage as described above.

In one preferred embodiment, the tumor is characterized by a lesion of at least about 3 mm in diameter, preferably at least 7 mm in diameter, and more preferably at least 1.5 cm in diameter.

In a sixth aspect the present invention relates to a vaccination kit comprising in separate packaging:
(i) a vaccine comprising the polypeptide of the first aspect of the invention, the nucleic acid of the second aspect of the invention, the vector of the third aspect of the invention or a collection of vectors according to the fourth aspect of the invention; and
(ii) at least one modulator of a checkpoint molecule or a nucleic acid encoding the modulator or a vector comprising the nucleic acid encoding the modulator.

### EXAMPLES

### Example 1: Fusion of Neo-Antigens to Either Invariant Chain or TPA Sequence Restores Immunogenicity in the Context of GAd Vaccination

A Great Ape Adenoviral vector encoding a pentatope containing 5 neo-antigens preceded by an initiator methionine (CT26-5; SEQ ID NO: 32) derived from the CT26 murine tumor is unable to induce an immune response against cancer antigens unless the INV sequence is placed at the N-terminus of the neo-antigens (CT26-5 INV; SEQ ID NO: 33). The ability to rescue immunogenicity was obtained as well by fusing a TPA sequence N-terminal to the string encoding the 5 neo-antigens (CT26-5 TPA; SEQ ID NO: 3).

The selected neo-antigens are generated by 5 non-synonymous single-nucleotide variants (SNVs), the most frequent type of mutations found in tumors. The amino acid sequence of each neo-antigen has the mutated amino acid placed in its center flanked both upstream and downstream by 12 wild-type (wt) amino acids for a total length of 25aa (table 1). Neo-antigen sequences are joined head to tail to form the artificial antigen fused downstream with an HA peptide sequence for the purpose of monitoring its expression (SEQ ID NO: 41).

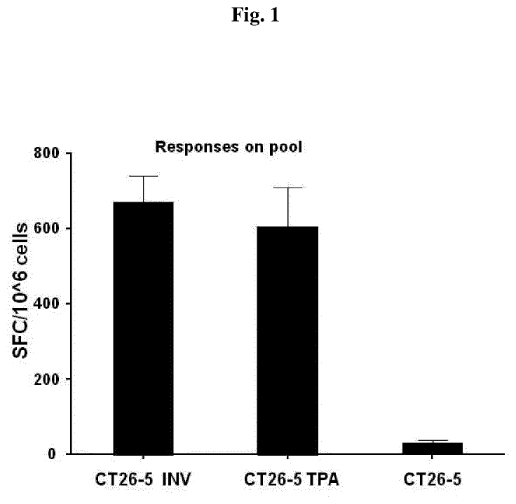

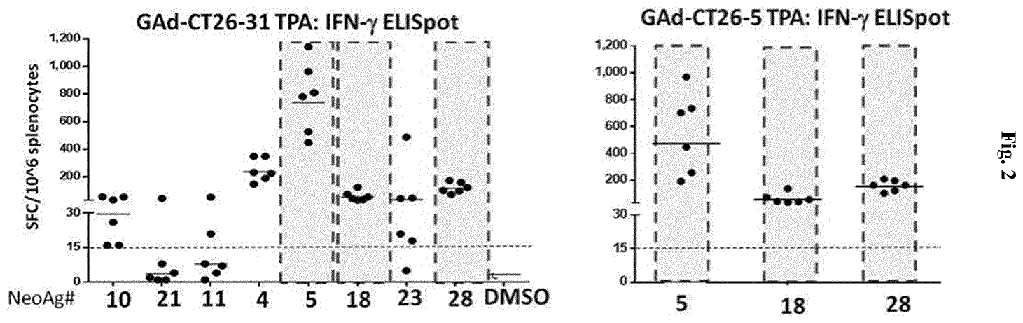

The immunological potency was evaluated in BalBC inbred mice after single intramuscular immunization at a dose of 5×10⁸ GAd viral particles (vp) for each of the 3 vaccines. Splenocytes were collected three weeks post-immunization and tested by IFN-γ ELISpot by stimulating cells in the presence of the pool of synthetic peptides corresponding to each of the 5 neo-antigens. Immune responses (number of T cells producing IFN-γ per million splenocytes) are shown in FIG. 1. Responses were considered positive if (i) the mean of antigen wells was greater than 20 Spot Forming Colonies SFC/10⁶ PBMC and (ii) exceeded by 3-fold the background value of wells incubated with the peptides diluent DMSO. As shown in FIG. 1, the addition of either INV or TPA converted the non-immunogenic CT26-5 antigen into an immunogenic antigen with 100% response rate in vaccinated animals.

### Example 2: A Large Number of Neo-Antigens is Required to Obtain a Synergic Activity Between the Vaccine and Anti-PD-1 in an Aggressive Therapeutic Setting

A second Great Ape Adenoviral vector (GAd-CT26-31 TPA) corresponding to a longer construct encoding for 31 neo-antigens with an N-terminal TPA sequence (CT26-31 TPA, SEQ ID 35) was constructed. The preferred TPA sequence used has the amino acid sequence of SEQ ID NO: 42. The selected mutations generating the neo-antigens are 31 non-synonymous SNV (Table 2), 3 of which were also present in the GAd-CT26-5 TPA vector encoding the shorter CT26-5 TPA construct (Table 1). The amino acid sequence of each of the 31 neo-antigens has the mutated amino acid placed in its center flanked both upstream and downstream by 12 wt amino acids for a total length of 25aa (Table 1). An exception is neo-antigen ID 6 (Table 2) where only 6 upstream wt amino acids are present corresponding to the N-terminus of the mutated protein and neo-antigen SEQ ID ID: 16 (Table 2) where an additional mutation generated by an additional SNV is present in the upstream amino acid segment (Table 2). The amino acid sequences of the neo-antigens were joined head to tail in the order shown in Table 2 and a HA peptide sequence (SEQ ID NO: 41) was added at the C-terminal end of the assembled neo-antigens for the purpose of monitoring expression.

Immunogenicity of GAd-CT26-5 TPA (short construct) and GAd-CT26-31 TPA (long construct) was determined in vivo by immunizing naïve BalbC mice intramuscularly once with a dose of 5×10⁸ viral particles (vp). T cell responses were measured 3 weeks post immunization by INF≡ ELISpot for recognition of individual peptides corresponding to the mutated 25mer sequences encoded by the vaccine constructs. The smaller construct (CT26-5 TPA) induced a T cell response only against 3 neo-antigens. Instead, vaccination with CT26-31 TPA induced T cell responses against 8 of 31 neo-antigens (FIG. 2) including the 3 neo-antigens shared with the CT26-5 construct.

To address whether the total number of immunogenic neo-antigens present in the vaccine represents a critical factor the inventors evaluated vaccination efficacy of the two constructs both in a prophylactic and in an aggressive therapeutic setting. In the prophylactic setting the inventors first vaccinated once with GAd-CT26-31 TPA or GAd-CT26-5 TPA (5×10⁸ vps/mouse) intramuscularly and subsequently, 15 days following vaccination, inoculated tumor cells (2×10⁶ cells per mouse). All vaccinated mice (100%) independently from the type of construct used were tumor-free while all control mice vaccinated with mock vaccine were sacrificed after 4 weeks because of the presence of very large tumors.

To mimic a therapeutic setting, BALB/c mice were engrafted with CT26 tumor cells (2×10⁶ cells per mouse). Tumor masses were measured over time and the treatment was started when the tumor mass became visible and reached a mean volume of 70 mm³. The therapeutic efficacy of GAd-CT26-31 TPA and GAd-CT26-5 TPA alone or in combination with an anti-PD1 antibody (clone RMP1-14, Bioxcell) treatment was then evaluated by initial treatment of established tumors with an intramuscular injection of a single dose of GAd-CT26-31 TPA or GAd-CT26-5 TPA vaccine (5×10⁸vps) and intraperitoneal injection of an anti-PD1 antibody. The anti-PD-1 antibody treatment was then continued for 2 weeks (days 3, 6, 9, 13, or 16).

Results showed that GAd-CT26-31 TPA and GAd-CT26-5 TPA vaccination without anti-PD1 antibody treatment was not effective in this setting and all mice were sacrificed after 4 weeks because of the presence of very large tumors like in untreated mice. Both vaccines were therefore unable to cure animals when used as standalone treatment, differently from what observed in the prophylactic setting. Anti-PD-1 monotherapy or combination of anti-PD-1 therapy with GAd-CT26-5 TPA vaccination caused tumor shrinkage in only 15% of treated mice. In contrast, combination of anti-PD-1 treatment with vaccination by GAd-CT26-31 TPA encoding the long construct provided remarkable anti-cancer activity with tumor shrinkage and complete cure in 48% of treated animals. Data are summarized in Table 3 showing that the difference between PD-1 monotherapy or PD-1/GAd-CT26-5 TPA combo relative to the PD-1/GAd-CT26-31 TPA combo is statistically significant. These results demonstrate that a genetic vaccine is capable of eradicating established tumors if encoding for a large number of neo-antigens and if combined with treatment by immunomodulatory molecules.

### Example 3: Comparison of Vaccination Efficacy in Three Different Settings

To address whether the number of neo-antigens present in the vaccine is critical to determine effectiveness of the vaccination approach, the inventors evaluated vaccination efficacy in three different settings: 1) a prophylactic setting, 2) an early intervention in a metastatic model of lung cancer and 3) advanced therapeutic setting of large established subcutaneous tumors.

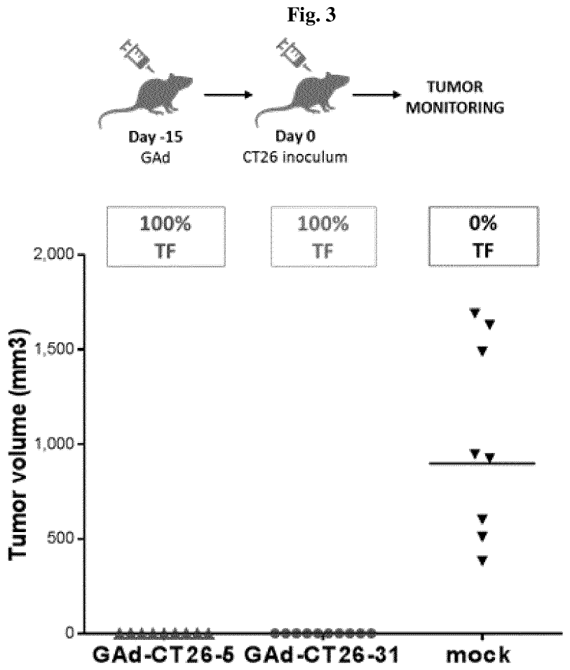

In a prophylactic intervention, mice were firstly immunized with GAd-CT26-31 or GAd-CT26-5 at the dose of 5×10\hat{ }8vp and two weeks later challenged with CT26 tumor cells to evaluate the preventative value of the vaccination. This led to protection from tumor take in 100% of vaccinated mice independently from the type of construct used, while all untreated mice developed large tumors (FIG. 3).

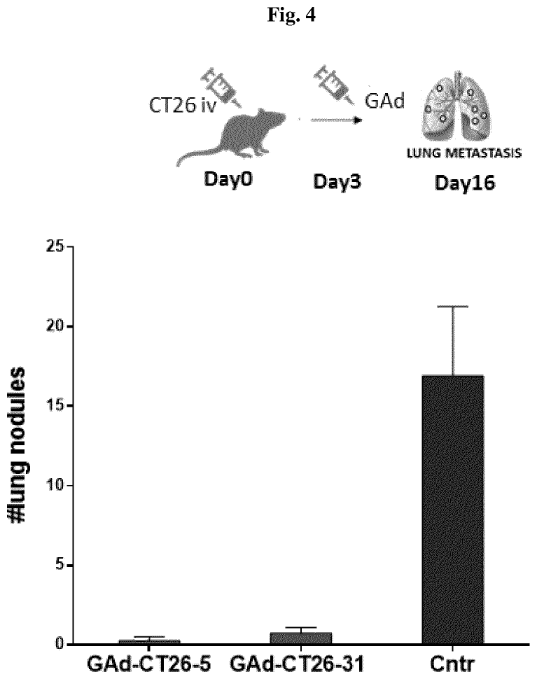

Similarly, GAd-CT26-31 and GAd-CT26-5 were equally effective in eradicating lung metastasis, measured by number of lung nodules, of CT26 cells in an early therapeutic setting, mimicking minimal residual disease because the tumor masses are not yet formed at time of vaccine delivery. The vaccination (dose of 5×10\hat{ }8 vp) was performed 3 days after the intravenous injection of tumor cells (FIG. 4).

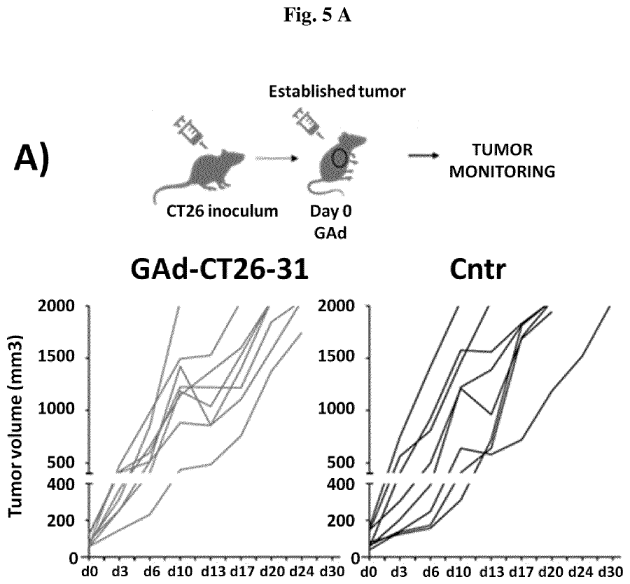

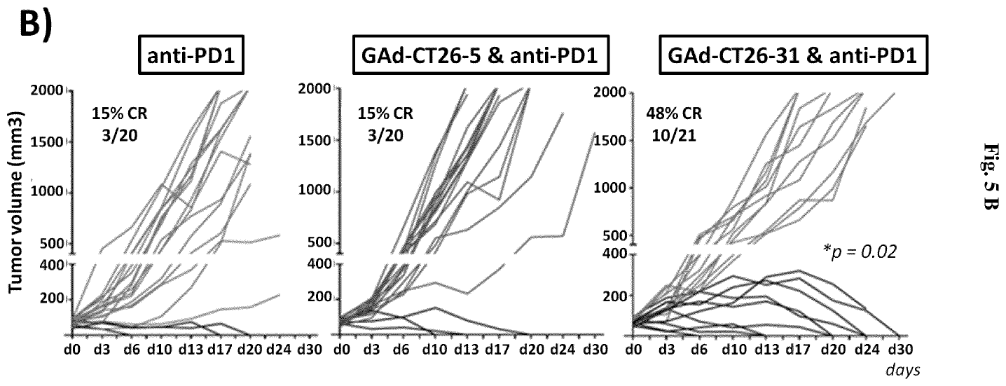

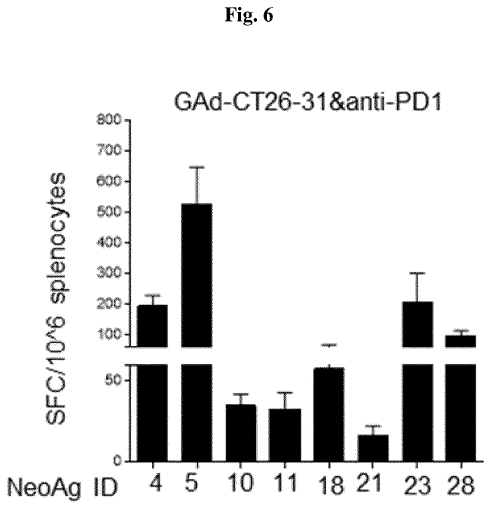

No anti-tumor activity was observed when mice bearing large established subcutaneous tumors were vaccinated GAd-CT26-31 TPA (FIG. 5A). A partial response was observed for anti-PD-1 monotherapy or a combination of anti-PD-1 therapy with GAd-CT26-5 TPA (FIG. 5b). Instead, a combination of PD1 blockade with the large construct GAd-CT26-31 TPA provided remarkable anti-cancer activity, leading to tumor shrinkage and complete cure in 48% of treated animals (FIG. 5B). Co-treatment with GAd-CT26-31 TPA and anti-PD1 induced T cell responses against the same 8 out of 31 neo-antigens (FIG. 6) compared to the vaccination with GAd-CT26-31 TPA alone in the prophylactic setting (FIG. 2).

### Example 4: Efficacy of the Personalized Vaccine is Dependent on the CD8+ T-Cell Response

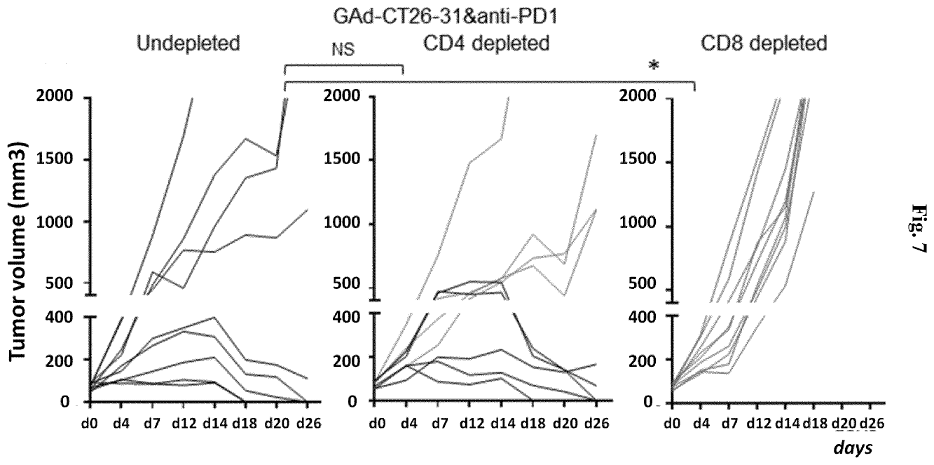

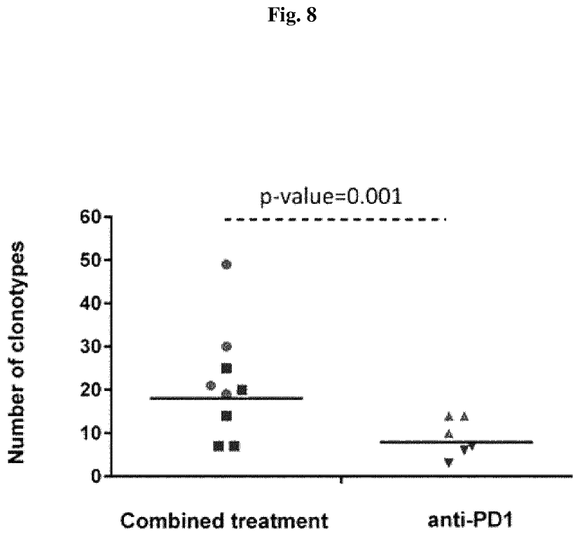

To investigate the contribution of CD4+ T cells and CD8+ T cells to the therapeutic antitumor effect of GAd-CT26-31 TPA, CD4+ or CD8+ T cells were depleted by specific antibodies (α-mCD8, BioXcell clone YTS169.4; α-mCD4, BioXcell clone YTS191) injected (200 μg) one week after the initiation of the therapy. CD8+ T cells depletion completely abrogated the anti-tumor effect (FIG. 8), highlighting the central contribution of CD8+ T cells. On the contrary, depletion of CD4+ T cells did not impact the efficacy of the treatment (FIG. 7). Identification of the CD8+ T-cell response as the mediator of efficacy is also in line with the known property of adenoviral vectors to generate a strong CD8+ T-cell response.

### Example 5: Efficacy of the Combined Personalized Vaccine and Anti-PD1 Treatment is Correlated with an Increase in TCR Clonality in the Tumor

RNA from CT26 tumors from mice treated only with anti-PD1 or treated by a combination of anti-PD-1 therapy with GAd-CT26-31 TPA was extracted and subjected to RNASeq analysis. Clonality of T-cell receptor (TCR) beta was assessed from the RNASeq data using the MIXCR tool applying the standard parameters reported in the RNA-seq workflow of the manual (https://mixcr.readthedocs.io/en/master/rnaseq.html). Raw output produced by MIXCR (sequences and expression of the detected TCR clonotypes) were further analyzed with the R package tcR (https://cran.r-project.org/web/packages/tcR/vignettes/tcrvignette.html) to obtain reconstructed CDR3 sequences and obtain summary statistics. As shown in FIG. 8 co-treatment with anti-PD-1 and GAd-CT26-5 TPA leads to the presence of a significantly higher number of distinct TCR clones (clonotypes) in tumors as compared to anti-PD1 treatment alone.

TABLE 1
CT26-5 individual neo-antigens:

| ID | Neo-antigen | Gene |
|---|---|---|
| 43 | LLPFYPPDEALEIGLELNSSALPPT | SLC4A3 |
| 5 | ILPQAPSGPSYATYLQPAQAQMLTP | E2F8 |
| 18 | KPLRRNNSYTSYIMAICGMPLDSFR | SLC20A1 |
| 28 | VIQTSKYYMRDVIAIESAWLLELAP | DHX35 |
| 44 | HIHRAGGLFVADAIQVGFGRIGKHF | AGXT2L2 |

The mutated amino acid is indicated in bold and underlined

TABLE 2
Individual neo-antigens present
in CT26-31:

| ID | Neo-antigens | Gene |
|---|---|---|
| 1 | PGPQNFPPQNMFEFPPHLSPPLLPP | PHF3 |
| 2 | GAQEEPQVEPLDFSLPKQQGELLER | ZEB1 |
| 3 | AVFAGSDDPFATPLSMSEMDRRNDA | TRAPPC12 |
| 4 | HSGQNHLKEMAISVLEARACAAAGQ | ALDH18A1 |
| 5 | ILPQAPSGPSYATYLQPAQAQMLTP | E2F8 |
| 6 | MSYAEKSDEITKDEWMEKL | GID8 |
| 7 | GAGKGKYYAVNFSMRDGIDDESYGQ | HDAC2 |
| 8 | YRGADKLCRKASSVKLVKTSPELSE | TTL |
| 9 | DSNLQARLTSYETLKKSLSKIREES | HAUS6 |
| 10 | HSFIHAAMGMAVTWCAAIMTKGQYS | NDC1 |
| 11 | LRTAAYVNAIEKIFKVYNEAGVTFT | GLUD1 |
| 12 | FEGSLAKNLSLNFQAVKENLYYEVG | G2E3 |
| 13 | DPRAAYFRQAENDMYIRMALLATVL | CAD |
| 14 | LRSQMVMKMREYFCNLHGFVDIETP | DARS2 |
| 15 | DLLAFERKLDQTVMRKRLDIQEALK | SMARCD1 |
| 16 | IKREKCWKDATYPESFHTLESVPAT | ZFP955B |
| 17 | GRSSQVYFTINVNLDLSEAAVVTFS | RWDD2B |
| 18 | KPLRRNNSYTSYIMAICGMPLDSFR | SLC20A1 |
| 19 | TTCLAVGGLDVKFQEAALRAAPDIL | DDX27 |
| 20 | IYEFDYHLYGQNITMIMTSVSGHLL | TOP3A |
| 21 | PDSFSIPYLTALDDLLGTALLALSF | SLC41A2 |
| 22 | YATILEMQAMMTLDPQDILLAGNMM | TTC39A |
| 23 | SWIHCWKYLSVQSQLFRGSSLLFRR | MTCH1 |
| 24 | YDNKGITYLFDLYYESDEFTVDAAR | SUV39H2 |
| 25 | AQAAKNKGNKYFQAGKYEQAIQCYT | TOMM70A |
| 26 | QPMLPIGLSDIPDEAMVKLYCPKCM | CSNK2B |
| 27 | HRGAIYGSSWKYFTFSGYLLYQD | CAPRIN2 |
| 28 | VIQTSKYYMRDVIAIESAWLLELAP | DHX35 |
| 29 | PRGVDLYLRILMPIDSELVDRDVVH | XPOT |
| 30 | QIEQDALCPQDTYCDLKSRAEVNGA | DCLRE1C |
| 31 | ALASAILSDPESYIKKLKELRSMLM | NOC3L |

The mutated amino acid is indicated in bold and underlined

TABLE 3

| | Number of induced | Frequency of | | | |
|---|---|---|---|---|---|
| Treatment(s) | T-cell reactivities | mice cured | | | |
| anti-PD1 | 1 | 15% | | | |
| GAd-CT26-5 & anti-PD1 | 3 | 15% | | | *p = 0.02 |
| | | | {close oversize bracket} | {close oversize bracket} | |
| GAd-CT26-31 & anti-PD1 | 8 | 48% | | | |

*Chi square test calculated on number of responders vs non-responders

1. A polypeptide comprising at least 25 different tumor-specific neo-antigens and at least one T cell enhancer amino acid sequence.

2. The polypeptide of claim 1, comprising at least 31 tumor-specific neo-antigens.

3. The polypeptide of claim 1, wherein each tumor-specific neo-antigens independently of each other have a length of 8 to 50 amino acids.

4. The polypeptide of claim 1, wherein each tumor-specific neo-antigen is independently selected from the group consisting of a single amino acid mutant peptide, a frame-shift peptide, a read-through mutation peptide, and a splice site mutant peptide.

5. The polypeptide of claim 1, wherein at least 4 of the tumor-specific neo-antigens elicits a T cell response in the patient.

6. The polypeptide of claim 1, wherein the tumor-specific neo-antigens are linked directly to each other.

7. The polypeptide of claim 1, wherein the T cell enhancer amino acid sequence is selected from the group consisting of an invariant chain; a leader sequence of tissue-type plasminogen activator (TPA); a PEST sequence; a cyclin destruction box; an ubiquitination signal; and a SUMOylation signal.

8. The polypeptide of claim 7, wherein:
(i) the TPA is an extended TPA leader sequence with an amino acid sequence according to SEQ ID NO: 42; and/or
(ii) the invariant chain is selected from the group consisting of:
(a) human invariant chain according to SEQ ID NO: 36, mouse invariant chain according to SEQ ID NO: 37, and Mandarin fish invariant chain according to SEQ ID NO: 38;
(b) an immune stimulatory fragment of an invariant chain according to (a); and/or
(c) an immune stimulatory variant of (a) or (b), wherein the variant has at least 70% sequence identity to the invariant chain according to (a) or a fragment thereof according to (b).

9. A nucleic acid encoding the polypeptide of any claim 1.

10. A vector comprising the nucleic acid of claim 9 operatively linked to an expression control sequence.

11. A collection of one or more expression vectors each comprising a nucleic acid according to claim 9, wherein each expression vector is selected from the group consisting of a plasmid; a cosmid; an RNA; an RNA-formulated with an adjuvant; an RNA formulated in liposomal particles; a self-amplifying RNA (SAM); a SAM formulated with an adjuvant; a SAM formulated in liposomal particles; a viral vector.

12. A composition comprising a vaccine comprising the polypeptide of claim 1 and at least one modulator of a checkpoint molecule or immunomodulator or a nucleic acid encoding the modulator or immunomodulator or a vector comprising the nucleic acid encoding the modulator or immunomodulator for use in preventing or treating a proliferative disease in a subject.

13. The composition of claim 12, wherein the modulator of a checkpoint molecule selected from the group consisting of:
(a) an agonist of a tumor necrosis factor (TNF) receptor superfamily member; and/or
(b) an antagonist of PD-1, PD-L1, CD274, A2AR, B7-H3 (e.g. MGA271), B7-H4, BTLA, CTLA-4, IDO, KIR, LAG3, TIM-3, or VISTA or an antagonist of a B7-CD28 superfamily member; and/or
(c) the other immunomodulator is a T cell growth factor like IL-2, IL-12, IL-15.

14. The composition of claim 12, wherein administration of the modulator of a checkpoint molecule is initiated before initiation of administration of the vaccine, or wherein administration of the checkpoint inhibitor is initiated after initiation of administration of the vaccine, or wherein administration of the checkpoint inhibitor is initiated simultaneously with the initiation of administration of the vaccine.

15. The composition of claim 12, wherein the subject is suffering from or is at risk of suffering from:
(a) Malignant neoplasms of lip, oral cavity and pharynx; and/or
(b) Malignant neoplasms of digestive organs; and/or
(c) Malignant neoplasms of respiratory and intrathoracic organs; and/or
(d) Malignant neoplasms of bone and articular cartilage; and/or
(e) Melanoma and other malignant neoplasms of skin; and/or
(f) Malignant neoplasms of mesothelial and soft tissue; and/or
(g) Malignant neoplasm of breast; and/or
(h) Malignant neoplasms of female genital organs; and/or
(i) Malignant neoplasms of male genital organs; and/or
(j) Malignant neoplasms of urinary tract; and/or
(k) Malignant neoplasms of eye, brain and other parts of central nervous system; and/or
(l) Malignant neoplasms of thyroid and other endocrine glands; and/or
(m) Malignant neoplasms of lymphoid, haematopoietic and related tissue.

16. The composition of claim 12, wherein the subject has a tumor according to the TNM classification of at least stage T1 with any stage of N and M, and/or a tumor that is characterized by a lesion of at least about 3 mm in diameter.

17. A vaccination kit comprising in separate packaging:
(i) a vaccine comprising the polypeptide of claim; and
(ii) at least one modulator of a checkpoint molecule or a nucleic acid encoding the modulator or a vector comprising the nucleic acid encoding the modulator.
